(19) United States
(12) Patent Application Publication
(10) Pub. No.: US 2022/0074616 A1
(43) Pub. Date: Mar. 10, 2022

(54) Air Cleaning and Purifying Apparatus for Elevators

(71) Applicant: Juan Jose Caravaca Hernandez, Murcia (ES)

(72) Inventor: Juan Jose Caravaca Hernandez, Murcia (ES)

(21) Appl. No.: 17/476,592

(22) Filed: Sep. 16, 2021

Related U.S. Application Data
(63) Continuation-in-part of application No. 17/468,283, filed on Sep. 7, 2021.

(30) Foreign Application Priority Data
Sep. 7, 2020 (ES) ES 202031949U

(51) Int. Cl. F24F 8/22; B66B 11/02; F24F 8/108; B01D 46/00; A61L 9/20

(52) U.S. Cl. CPC F24F 8/22; B66B 11/024; F24F 8/108; B01D 46/0038; B01D 46/0024; B01D 46/0028; B01D 2279/40; A61L 9/205; A61L 2209/14; A61L 2209/16; B01D 2267/40; B01D 2267/70; B01D 46/008

(57) ABSTRACT

An air cleaning/purifying system for elevators includes: a housing, one or more fans, three filters, and a lamp. The housing, made of a chromium-nickel-molybdenum austenitic stainless steel, is formed of one or more walls that create an air tight enclosure, except for one or more air inlet openings proximate to each fan, and a plurality of air outlet openings. The three filters are positioned between the inlet and outlet openings, and include: an active carbon filter to absorb odors; a HEPA filter formed of a fiber mesh that traps particles; and a filter formed of a selective metallic compound (e.g., titanium dioxide) that emits hydroxyl radicals when illuminated by light from the lamp. The fan(s) is/are mounted to the housing and positioned to draw/direct air into the air tight enclosure through the inlet opening(s) and direct it at the filters, in the direction of the outlet openings.

### CROSS REFERENCES TO RELATED APPLICATIONS

This application is a continuation in part of U.S. patent application Ser. No. 17/468,283, filed on Sep. 7, 2021, which claims priority on Spanish Patent Application Ser. No. ES202031949U, filed on Sep. 7, 2020, all disclosures of which are incorporated herein by reference.

### FIELD OF THE INVENTION

The subject technology relates generally to air purification devices, and more particularly to air purification and sanitizing devices that are adapted for use in elevators and/or other small spaces.

### BACKGROUND OF THE INVENTION

Elevators generally lack any built-in air purification apparatus or mechanism. Elevators only afford a very small space through which people, pets, luggage and cargo move frequently, generating and retaining smells, and germs of all kinds within that space. Since the elevator doors are normally closed, and are only opened periodically for a brief amount of time, they become a hazardous repository of dust, pollen and other particulates, bacteria, mold, viruses, and other contagions. Therefore, an affirmative and highly effective system is necessary to eradicate the health hazard.

While air cleaners tend to be directed to and efficient at the removal of dust, pollen, and other particulates, sanitizers/purifiers tend to be directed to killing mold, bacteria, etc. Thus, there is an unmet need for an improved and highly effective system that addresses all aspects of air quality within the closed environment of an elevator.

Devices/methods that may be related, and which are not admitted herein to be prior art to the presently disclosed apparatus, may be shown by the following: U.S. Pat. No. 5,616,172 to Tuckerman for an “Air Treatment System”; U.S. Pat. No. 5,997,619 to Knuth for “Air Purification System”; U.S. Pat. No. 6,613,277 to Monagan for an “Air Purifier”; U.S. Pat. No. 7,323,146 to Kim for an “Air Purifier”; U.S. Pat. No. 7,674,436 to Feldman for “Portable Indoor Air Purification System”; U.S. Pat. No. 8,398,917 to Itzhak for “Method and Apparatus for Treating Biologically Contaminated Air”; U.S. Pat. No. 10,875,744 to Doyle for “Passenger Elevator Air Purification System”; U.S. Patent App. Pub. No. 2015/0202107 to Khan for “Air Purification System for Operating Theatres”; U.S. Patent App. Pub. No. 2019/0366263 to Zhu for “Method and Apparatus for Purification and Treatment of Air”; and U.S. Patent App. Pub. No. 2020/0129972 to Ozaki.

The present invention is different than certain prior art inventions in many ways, including, but no limited to, the fact that the present invention does not electrically charge dust particles by corona discharge, so that they may be collected by electrostatic attraction using an electrostatic filter having a polarity opposite to the polarity of the charged dust particles.

It is noted that the citing of any reference within this disclosure, i.e., any patents, published patent applications, and non-patent literature, is not an admission regarding a determination as to its availability as prior art with respect to the herein disclosed and claimed apparatus.

### OBJECTS OF THE INVENTION

It is an object of the invention to provide an apparatus for treating and improving the air quality within a small enclosed space, such as an elevator.

It is another object of the invention to provide an improved apparatus for purifying and sanitizing air within an enclosed space.

It is a further object of the invention to provide an improved apparatus for filtering dust, pollen, and other particles from an enclosed space, such as an elevator.

It is another object of the invention to provide an improved apparatus for filtering odors from an enclosed space, such as an elevator.

It is also an object of the invention to provide an improved apparatus with a germicidal mechanism to eliminate fungi, spores of fungi, bacteria, viruses, yeasts, protozoa, etc. in an enclosed space, such as an elevator.

Further objects and advantages of the invention will become apparent from the following description and claims, and from the accompanying drawings.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

The apparatus disclosed herein is conceived and adapted to be incorporated for use with respect to an elevator car, although its use in other enclosed areas is also contemplated, such as for a hospital room, a room in a home, etc. The apparatus disclosed herein is specially designed to accomplish sterilization of air, and prevents the reproduction of bacteria, viruses, fungi, yeasts protozoa, etc., thereby preserving the environment. Microbes and other pathogens are vulnerable to the effects of ultraviolet light at certain wavelengths, particularly at 253.7 nanometers. Thus, the apparatus disclosed herein acts as a sterilizer, affecting the molecular structure of any microorganisms contained within the air in the enclosed space (i.e., the elevator car) and within the housing of the device, eliminating the microorganisms and preventing its reproduction.

In this way, the air sterilizing device for elevators provides a solution for the sanitization of air and elimination of ethylene in elevators and in environments prone to the existence of bacteria and microorganisms.

The herein disclosed apparatus incorporates a series of filters, which purify the air without leaving residues, and are not toxic or harmful to health, and neither are they harmful to humans or animals. In addition, the present apparatus eliminates odors and volatile organic compounds (VOCs), which are normally generated in confined spaces such as elevator cars.

Thus, among the technical advantages offered by the herein disclosed air sterilizing device for elevators, are the following.

The treatment provided by the device does not produce chemical residues. The device is effective for the disinfection of various surfaces. The equipment is effective for the inactivation of multiple microorganisms, being simple and easy to handle and install. The device does not involve a high manufacturing cost or maintenance cost. The optimum temperature range for the correct operation of the herein disclosed device is from about −5° C. to 40° C.

Another outstanding feature of the apparatus, is that its entire structure is made of 316L surgical stainless steel. It is an austenitic chrome nickel stainless steel containing molybdenum, which increases overall corrosion resistance, improves resistance to chloride ion solution pitting, and provides greater resistance to degradation at elevated temperatures.

The herein disclosed apparatus includes one or more fans, whose function is to force the circulating air flow towards the filters. The apparatus incorporates a HEPA filter, composed of a fiber mesh, whose main function is to trap pathogenic particles or elements. It also incorporates an active carbon filter, whose main function is to eliminate odors. The herein disclosed apparatus also includes a germicidal mechanism to eliminate the spores of fungi and bacteria that are often present in the air flow of such enclosed spaces. In addition, a titanium dioxide filter is incorporated that, together with the ultraviolet light from the germicidal lamp, generates hydroxyl radicals that allow the sterilization of the air that circulates through the device, purifying the air in the spaces where it is utilized.

The three filter utilized (i.e., HEPA, activated carbon, and titanium) are capable of eliminating 99.97% of the pollutants. Thus, the herein disclosed apparatus when installed with respect to an elevator car, provides optimal air quality for passengers of the elevator. In addition, the apparatus is also environmentally friendly, since the filters it incorporates purify the atmosphere without leaving residues, they are not toxic, and are not harmful to health.

In summary, in order to solve the problem of air quality associated with elevator cars and other small spaces, and provide excellent air quality, without odors, microbes, bacteria, viruses, etc., the herein disclosed air sterilizing device includes: a container casing; one or more fans whose function is to force the circulating air flow towards and through the filters; a HEPA filter composed of a mesh of fibers, whose main function is to trap pathogenic particles or elements; an active carbon filter whose main function is to eliminate odors; a germicidal mechanism with ultraviolet light to eliminate the spores of fungi and bacteria, which are in the air flow, which can penetrate the described apparatus; a titanium filter, which together with the ultraviolet light from the germicidal lamp, generates hydroxyl radicals, which molecules allow the sterilization of the air that circulates through the device, purifying the air in the enclosed space.

### BRIEF DESCRIPTION OF THE DRAWINGS

The description of the various example embodiments is explained in conjunction with appended drawings, in which:

FIG. 1 is a schematic profile view of a first embodiment of an apparatus for improved air cleaning and sterilizing in an enclosed space such as an elevator,

FIG. 2 is a side view of a second embodiment of an apparatus for improved cleaning and sterilizing of the air of an enclosed space such as an elevator,

FIG. 2A is a side view of an elevator car, shown with the air cleaning and sterilizing apparatus of FIG. 2 mounted to a surface of the elevator car to act upon and provide cleaned and sterilized air thereto;

FIG. 3 is a photo of the interior of a prototype of the apparatus of FIG. 2, shown with three fans and electronics installed in the housing;

FIG. 4 is another photo of the prototype of FIG. 3, shown with a filter and UV light bulbs also installed in the housing;

FIG. 5 is another photo of the prototype of FIG. 4, shown with an additional filter installed in the housing, and with the UV light bulbs lit up;

FIG. 6 is another photo of the prototype of FIG. 5, shown enlarged;

FIG. 7 is a photo showing the on/off switch and air inlet openings to the fans on one side of the housing of the prototype of FIG. 5;

FIG. 8 is a photo showing another side of the housing of the prototype of FIG. 5;

FIG. 9 is an enlarged detail view of the interior of the prototype of FIG. 5;

FIG. 10 is a second enlarged detail view of the interior of the prototype of FIG. 5;

FIG. 11 is a third enlarged detail view of the interior of the prototype of FIG. 5;

FIG. 12 is a perspective view of a filter used in the prototype of FIG. 5;

FIG. 13 is a photo showing a first perspective view of another prototype similar to the apparatus of FIG. 2, but having only one fan, and corresponding inlet openings on the housing;

FIG. 14 is a photo showing a second perspective view of the prototype of FIG. 13;

FIG. 15 is a photo showing a side view of the prototype of FIG. 13;

FIG. 16 is a photo showing a bottom view of the prototype of FIG. 13;

FIG. 17 is a side view of a third embodiment of an apparatus for improved cleaning and sterilizing of the air of an enclosed space such as an elevator, shown with the cover plate removed;

FIG. 17A is a cover plate used to seal an access opening in the housing of the apparatus of FIG. 17;

FIG. 18 is the side view of FIG. 17, but shown with the filter pack removed;

FIG. 19 is a side view of the filter assembly used in the apparatus of FIG. 17, shown prior to being installed in the apparatus of FIG. 18;

FIG. 20 is a top view of the filter assembly of FIG. 19;

FIG. 21 is a top view of the coarse air filter used in the filter pack assembly of FIG. 19;

FIG. 22 is a top view of the active carbon filter used in the filter pack assembly of FIG. 19;

FIG. 23 is a top view of the HEPA filter used in the filter pack assembly of FIG. 19; and

FIG. 24 is a top view of the metal oxide filter used in the filter pack assembly of FIG. 19.

### DETAILED DESCRIPTION OF THE INVENTION

As used throughout this specification, the word “may” is used in a permissive sense (i.e., meaning having the potential to, or being optional), rather than a mandatory sense (i.e., meaning must), as more than one embodiment of the invention may be disclosed herein. Similarly, the words “include”, “including”, and “includes” mean including but not limited to.

The phrases “at least one”, “one or more”, and “and/or” may be open-ended expressions that are both conjunctive and disjunctive in operation. For example, each of the expressions “at least one of A, B and C”, “one or more of A, B, and C”, and “A, B, and/or C” herein means all of the following possible combinations: A alone; or B alone; or C alone; or A and B together; or A and C together, or B and C together; or A, B and C together.

Also, the disclosures of all patents, published patent applications, and non-patent literature cited within this document are incorporated herein in their entirety by reference. However, It is noted that the citing of any reference within this disclosure, i.e., any patents, published patent applications, and non-patent literature, is not an admission regarding a determination as to its availability as prior art with respect to the herein disclosed and claimed apparatus.

Furthermore, any reference made throughout this specification to “one embodiment” or “an embodiment” means that a particular feature, structure or characteristic described in connection therewith is included in at least that one particular embodiment. Thus, the appearances of the phrases “In one embodiment” or “in an embodiment” in various places throughout this specification are not necessarily all referring to the same embodiment. Therefore, the described features, advantages, and characteristics of any particular aspect of an embodiment disclosed herein may be combined in any suitable manner with any of the other embodiments disclosed herein.

Additionally, any approximating language, as used herein throughout the specification and claims, may be applied to modify any quantitative or qualitative representation that could permissibly vary without resulting in a change in the basic function to which it is related. Accordingly, a value modified by a term such as “about” is not to be limited to the precise value specified, and may include values that differ from the specified value in accordance with design variations described in the specification, as well as applicable case law. Also, in at least some instances, a numerical difference provided by the approximating language may correspond to the precision of an instrument that may be used for measuring the value. A numerical difference provided by the approximating language may also correspond to a manufacturing tolerance associated with production of the aspect/feature being quantified. Furthermore, a numerical difference provided by the approximating language may also correspond to an overall tolerance for the aspect/feature that may be derived from variations resulting from a stack up (i.e., the sum) of a multiplicity of such individual tolerances.

It is further noted that any use herein of relative terms such as “top,” “bottom,” “upper,” “lower,” “vertical,” and “horizontal” are merely intended to be descriptive for the reader, and may be based on the depiction of those features within the figures for one particular position of the apparatus, and such terms are not intended to limit the orientation with which the disclosed apparatus may be utilized.

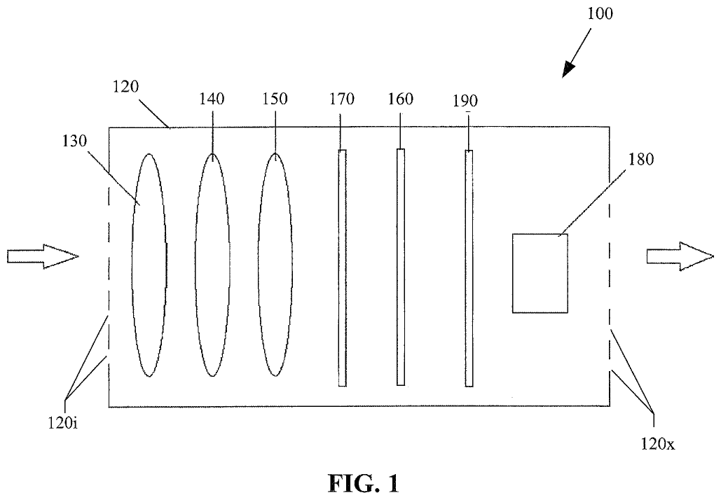

FIG. 1 shows a schematic side view of an air sterilizing device 100, which is particularly configured to improve multiple different aspects of the air quality associated with elevator cars and other small spaces, and provide excellent air quality, being without odors, microbes, bacteria viruses, etc.

The device 100 may broadly include: a housing 120; one or more fans 130, 140, and 150, which may draw air in through a plurality of inlet openings 1201 into the housing enclosure, and direct the air within the housing to force the circulating air flow towards a series of filters 160, 170, and 190. For the device 100, the air directed by the fan(s) 130/140/150 may be directed towards, and be initially filtered by, the filter 170, which may be a HEPA filter composed of a mesh of fibers. The main function of filter 170 is to trap particulates. The filter 170, as well as filters 160 and 190, may each extend across an entire interior periphery of the interior of the housing 120, so that the air passing through the housing interior as a result of the fan(s) must pass through each of the series of filters. Once the air has passed through the first filter 170, it is next driven through the second filter 160, which is an active carbon filter. The main function of the second filter 160 is to eliminate odors. Once the air has passed through the second filter 160, it is next driven through the third filter 190, which is formed to include titanium dioxide. The titanium dioxide of filter 190 in combination with the light from ultraviolet lamp 180 co-act to create a germicidal mechanism that eliminates spore, the spores of fungi, bacteria, etc. The titanium dioxide of filter 190 when illuminated by the ultraviolet light from the germicidal lamp 180 generates hydroxyl radicals, which allow the sterilization of the air that circulates through the device, purifying the air in the enclosed space. The ultraviolet lamp 180 is positioned between the filter 190 and the air outlet/exit openings 120x.

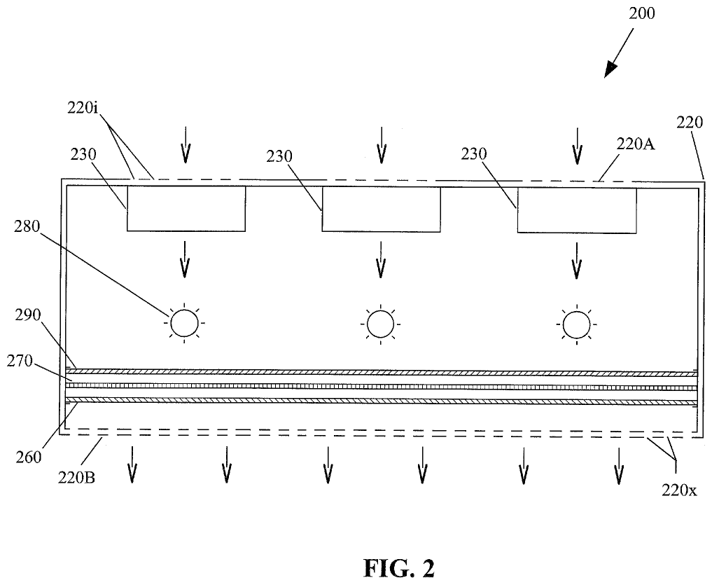

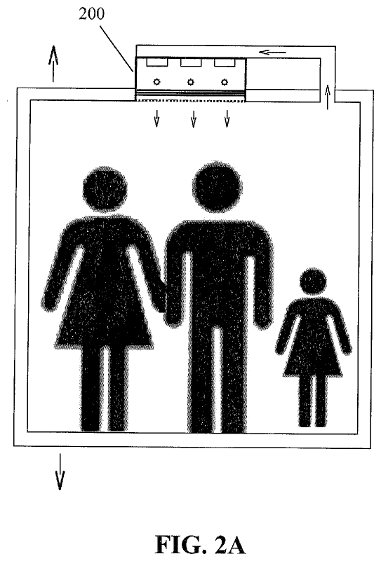

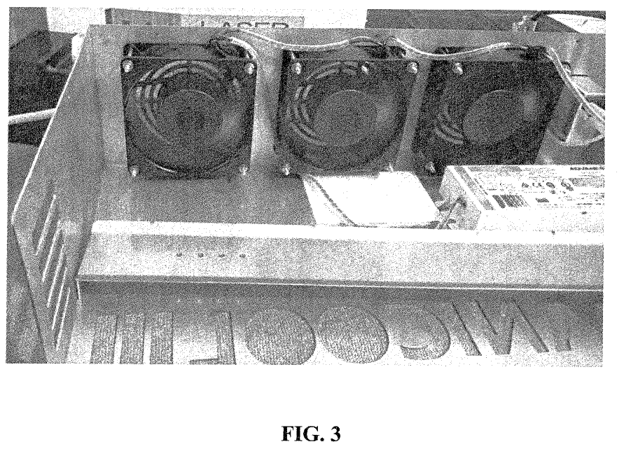

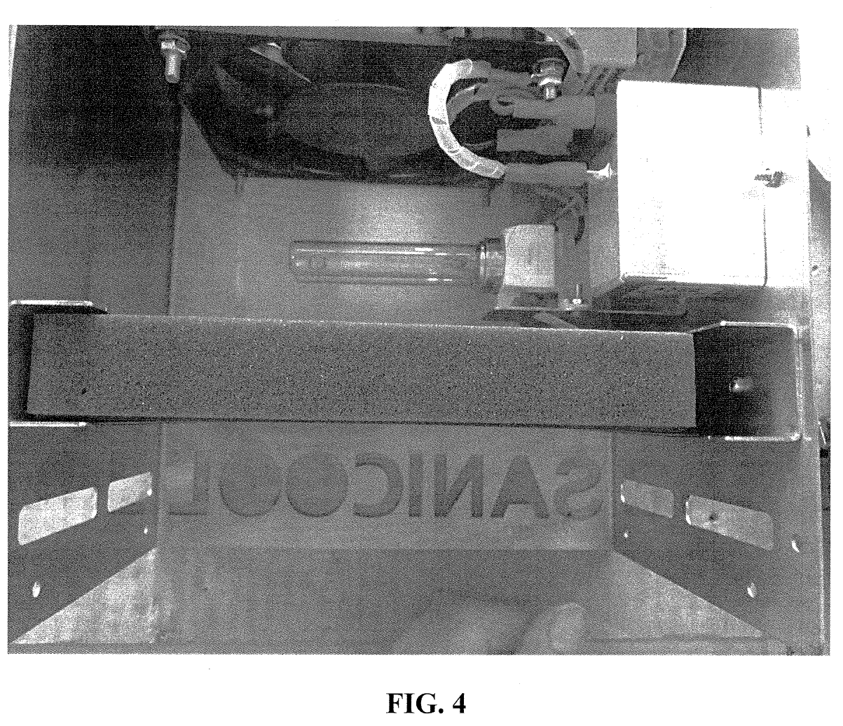

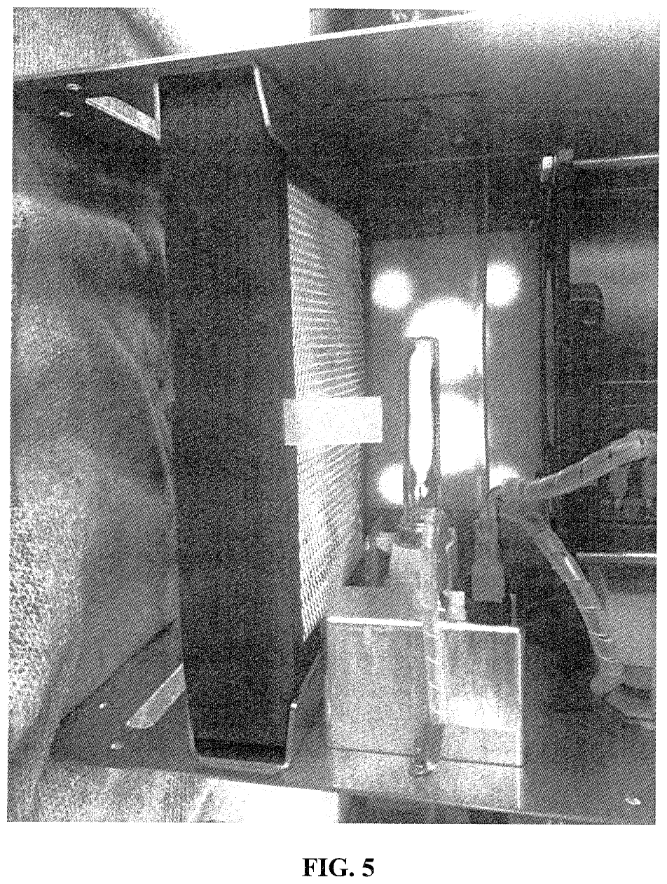

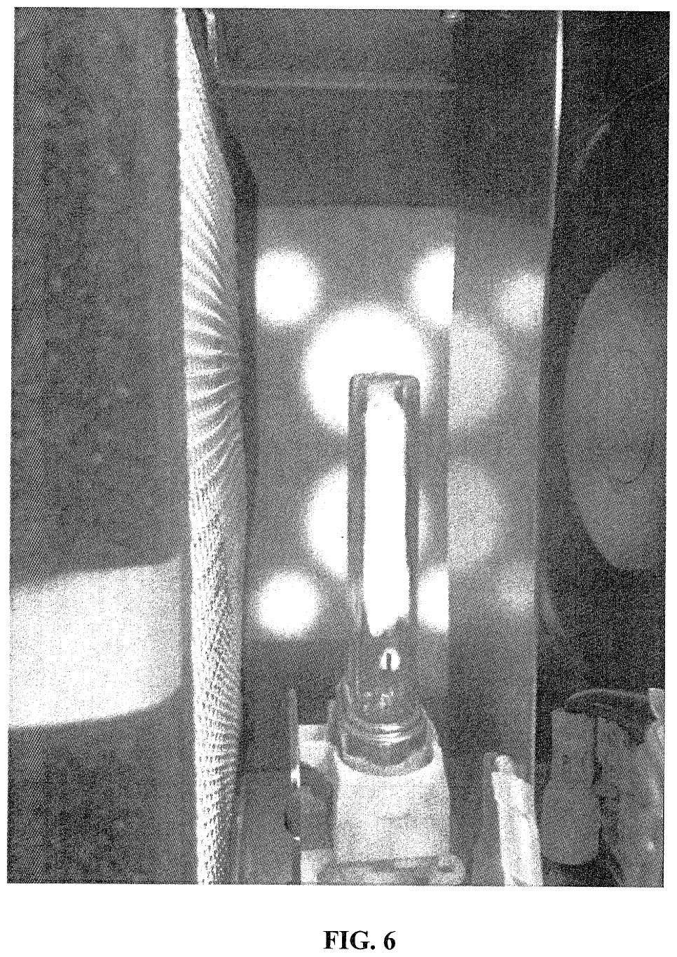

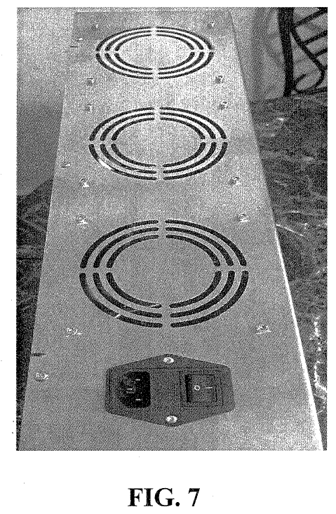

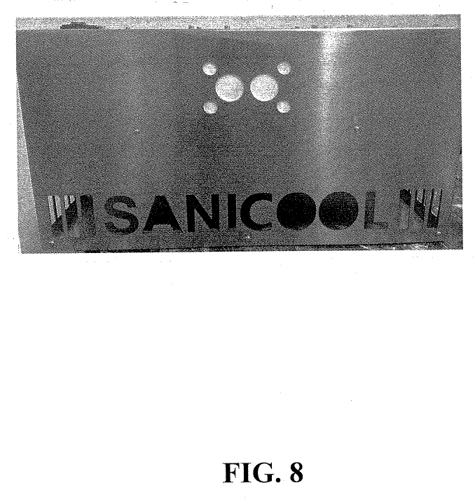

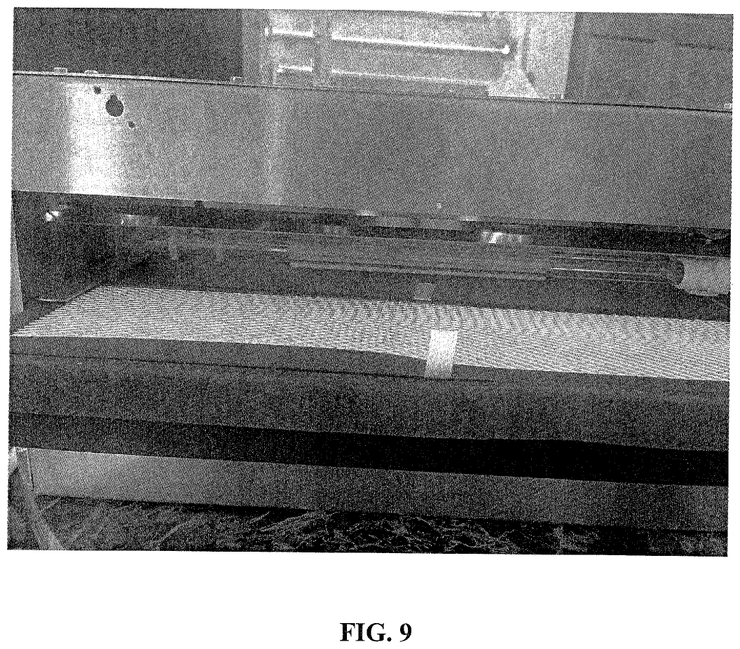

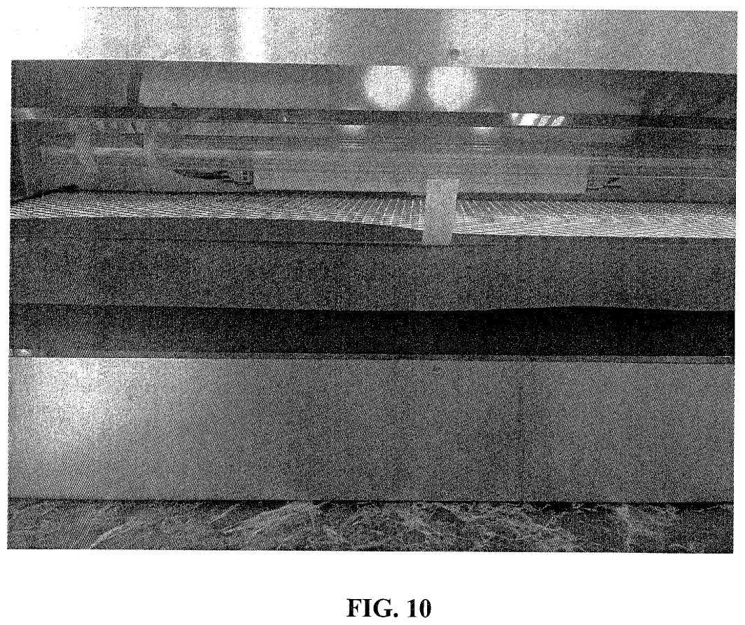

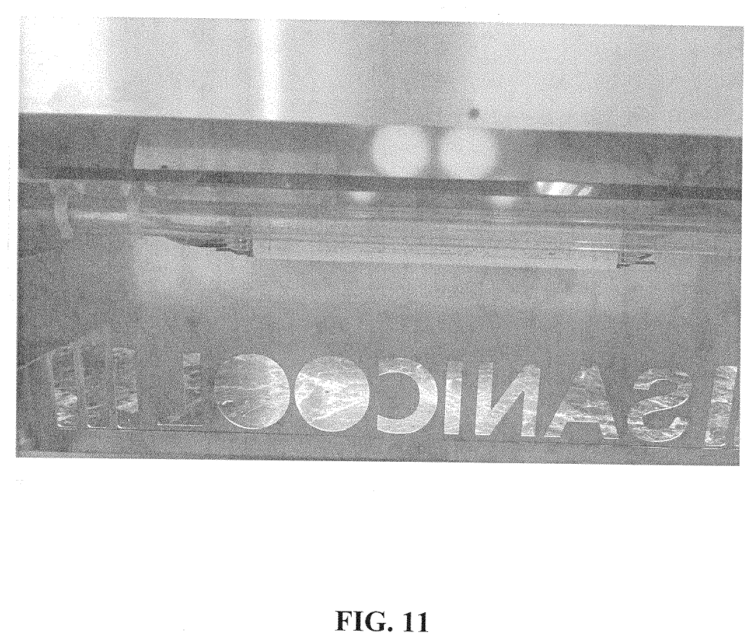

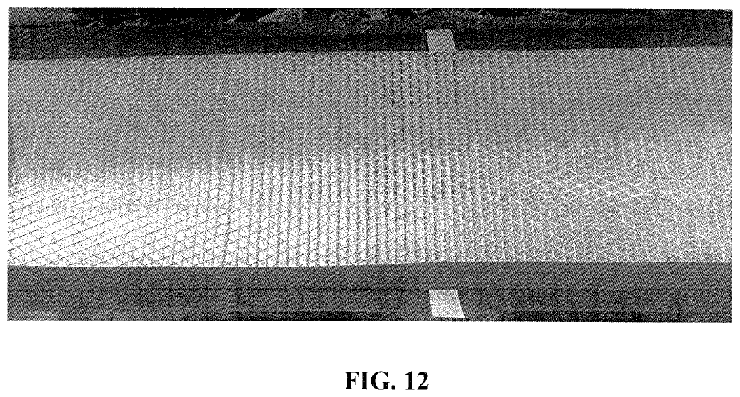

FIG. 2 shows a side view of an apparatus 200 that is particularly configured to improve multiple different aspects of the air quality associated with elevator cars and other small spaces, by both cleaning the air through removal of particulates, and purifying the air with respect to killing microorganisms, viruses, etc., that may be present. The apparatus 200 may include: a housing 220 that may be formed of one or more walls to create a substantially air tight enclosure; one or more fans 130; one or more lamps 280; and three different filters—filter 290, filter 270, and filter 260.

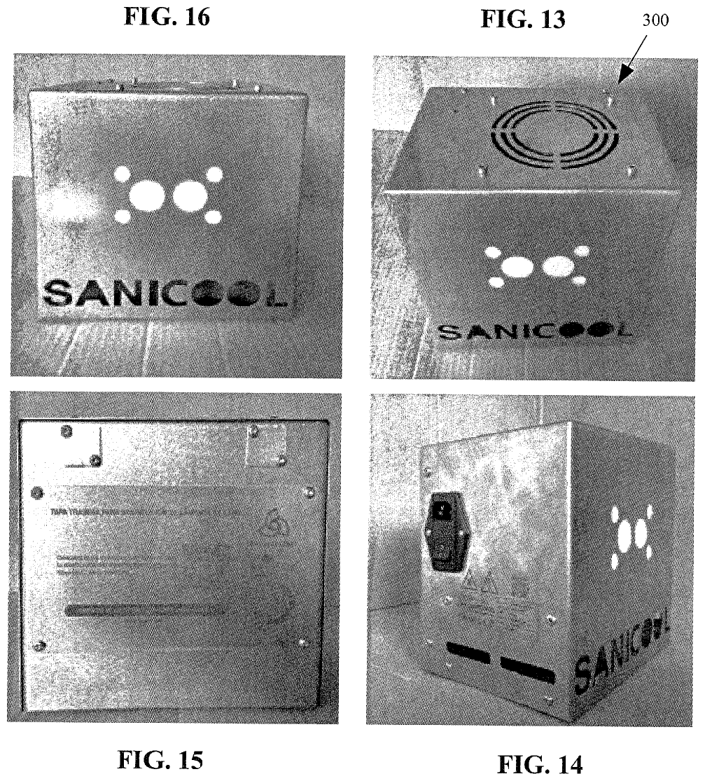

The one or more walls that form housing 220 may create a rectangular-shaped enclosure, as seen in FIG. 2, or alternatively, in another housing embodiment, the one or more walls may create a square box-shaped enclosure (see e.g., device 300′ shown in FIGS. 13-16). In yet another housing embodiment, the housing may be cylindrical in shape, and may thus eliminate any interior corners, in which embodiment certain electronics and associated components may be mounted on the exterior of the housing, as the housing interior may be so constructed to only accommodate airflow through the various filters. Other housing shapes may be used in other embodiments. For any of these shapes, the housing 220 may preferably be formed of 316L (surgical) stainless steel, which is a chromium-nickel-molybdenum austenitic stainless steel developed to provide improved corrosion resistance, including at elevated temperatures

The otherwise substantially air tight enclosure formed by the one or more walls of housing 220 may have a plurality of air inlet openings 2201 formed in a first wall portion 220A of the housing, and may also have a plurality of air outlet/exit openings 220x formed in a second wall portion 220B. The first wall portion 220A and the second wall portion 220B may each be substantially planar, and may be substantially parallel to each other.

When one fan 230 is used, the air inlet openings 2201 may be formed into a single grouping in an area that is located just in front of the fan (see e.g., FIG. 13). The housing of the fan may be sealed with respect to the housing 220, so the only air path provided by the air inlet openings 2201 may be through the fan, which may result from its rotating blades. Where multiple fans 230 are used, i.e., the three fans shown in FIG. 2, there may be three groupings of the air inlet openings 2201, one grouping in front of each fan. It is noted that the fans, although shown mounted to the interior of the housing 220, may alternatively be mounted on its exterior.

This arrangement—a grouping of air inlet openings 2201 being positioned in front of each fan that is mounted to the first wall portion 220A—permits each fan to direct/draw air into the enclosure through the corresponding air inlet openings, and be directed generally towards the plurality of air outlet openings.

The first filter of apparatus 200 that the air from the fan(s) 230 may strike is filter 290. Filter 290 may be formed of, or be formed to include, a selective metal oxide compound (e.g., titanium dioxide—TiO2), which undergoes a photocatalytic effect when exposed to the light from the lamp(s) 280, and thereby generates hydroxyl radicals (i.e., OH molecules) that sterilize the air. The sterilization includes causing the decomposing of organic matter (e.g., fungi, spores of fungi, bacteria, viruses, yeasts, protozoa, etc.), into harmless substances such as carbon dioxide and water.

The fan(s) 230 may be mounted to the first wall portion 220A to direct the air substantially perpendicularly to, i.e., directly at, the filter 290, and may thereby create pressure to cause air to flow through the series of filters 290/270/260, each of which may be substantially parallel to the first wall portion. Also, the series of filters 290/270/260 may be mounted in the housing 220 such that there is a gap between each adjacent filter, as shown in FIG. 2, or they may be mounted to contact the adjacent filter.

Each of the lamps 280 may include, but is not limited to, a low pressure mercury lamp, an excimer lamp, a pulsed xenon lamp, and a light emitting diode. Also, a combination of such lamps may also be used. The lamp or lamps 280 may be mounted anywhere within the interior of the housing 220 at a position that is between the first wall portion 220A and the metal oxide filter 290, to illuminate that filter and facilitate the photocatalytic effect. In one embodiment, each of three lamp 280 may be mounted to be centrally positioned with respect to a corresponding one of the three fans, and may thereby be centrally positioned in the air that is directed by the respective fan towards the filter 290, as shown in FIG. 2. This arrangement may further enhance the germicidal effect of the light emitted by the lamp(s) 280, which may also directly act upon the air, through the use of ultraviolet wavelengths that directly kills fungi, spores of fungi, bacteria, viruses, yeasts and protozoa.

The wavelength emitted by the lamps 280 may be in the range of 100 nanometers to 400 nanometers; however, UV-C light (i.e., wavelengths between 200 nm and 280 nm) has been shown to be particularly effective at disinfection. In another embodiment, each lamp may provide a dose of 40 mJ-cm² of 254 nm light, which may kill about 99.9% of the pathogenic organisms present. UVC light is also effective at killing the corona virus. The 265 nm wavelength is also particularly effective at disabling other viruses. Furthermore, the 222 nm wavelength is believed to damage proteins on the surface of a virus that it uses to attach itself to human cells. Therefore, in another embodiment, each lamp 280 may preferably emit a spectrum of UV-C light that includes two or three of these wavelengths (i.e., 222 nm, 254 nm, and 265 nm). For example, in one embodiment a spectrum of light between and including 222 nm to 265 nm may be used. In another embodiment a spectrum of light between and including 254 nm to 265 nm may be used.

In one embodiment, the apparatus 200 may utilize one lamp 280 for the entire enclosure, and may use an arrangement of three fans 230 as shown to blow air throughout the conduit. In another embodiment, one lamp 280 may be used for each fan of the three-fan arrangement. The bulb portion of the lamp may be sized to stretch across nearly the entire linear extent of the interior. In other embodiments, other numbers of lamps, and sizes of the lamps may be utilized, such that they illuminate the interior of the enclosure of the housing 220, and properly illuminate the metal oxide filter 290.

The filter 270 may be an active carbon filter configured to absorb odors, and the filter 260 may be a HEPA filter formed of a fiber mesh configured to trap particles, and each may be positioned as shown in FIG. 2. The light emitted by the lamp(s) 280 also operate to sterilize the HEPA filter 260 and thus prevents it from being contaminated with the particles it retains.

With the three filters 290/270/260 positioned as shown in FIG. 2 for the apparatus 200, a small cavity may be formed between the fan(s) 230 and the filter 290, and the air directed into the cavity may circulate therein, particularly when the lamp is positioned directly in the airstream, and/or the air may at least spend some time in the cavity. The air while in the cavity may be disinfected by the combination of the UV light acting directly upon it, and by the hydroxyl radicals emitted by the titanium dioxide filter.

To cause the air to be forced through each of the series of filters 290/270/260, each filter is shaped to extend across an entire interior periphery of the housing 220. It is noted that a different sequence may be used for the positioning of the three different filters, along with a corresponding placement of the lamp(s) 280.

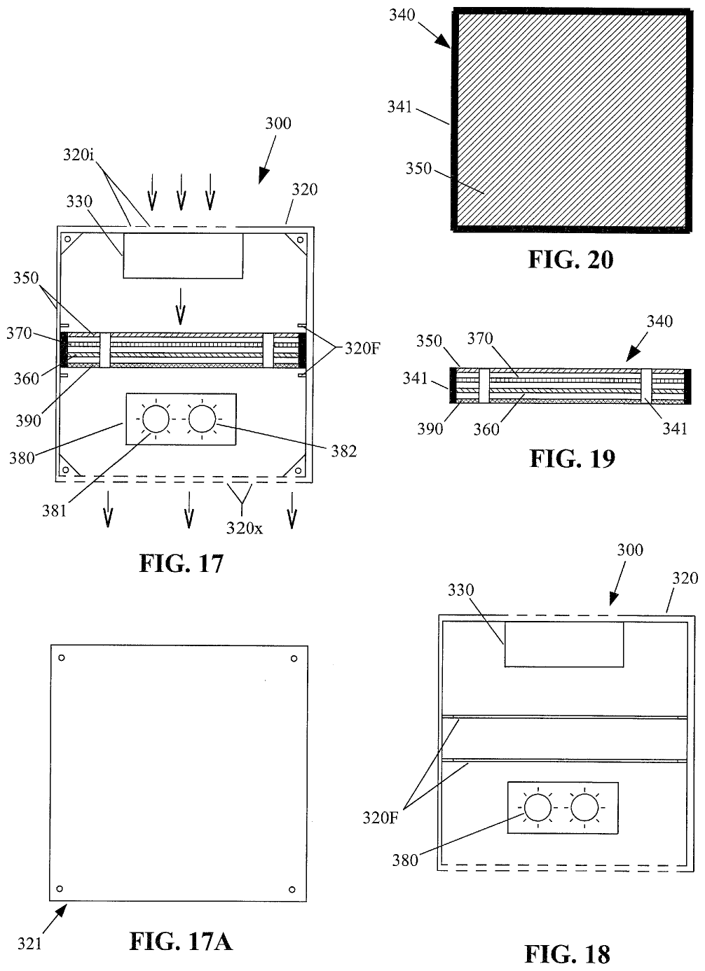

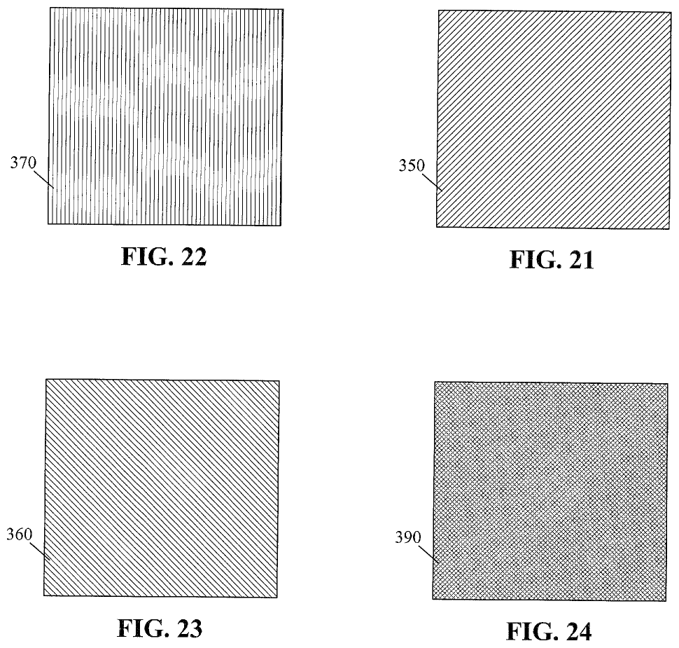

In another embodiment, shown in FIG. 17, an apparatus 300 may use a housing 320, being similar to the housings previously described, having an access opening that may be sealed using a cover plate 321 (FIG. 17A); one or more fans 330 to blow air throughout the housing conduit; and a lamp 380 that may utilize a dual bulb arrangement (i.e., bulbs 381 and 382) to redundantly illuminate the metal oxide filter 390 (i.e., should one bulb fail, the other bulb will still enable the photocatalytic effect). For the apparatus 300, as well as the other embodiments, the different filters may be bound together into a filter pack assembly 340, as seen in FIGS. 19-20. The filter pack 340 may include, in sequence, a coarse filter 350 (see FIG. 21), which is configured to remove large particles (e.g., particles over one micron), an active carbon filter 370 (see FIG. 22) that is configured to absorb odors, a HEPA filter 360 (see FIG. 23) configured to trap 99.997% of all particles larger than 0.1 microns, and the metal oxide filter 390 (see FIG. 24).

The carbon filter 370 is unique, as it is impregnated with organic essential oils from plants that operates to eliminate bacteria of all kinds, being effective also for fungi, parasites and viruses. It has no side effects of any kind. The active ingredient in the organic essential oils is carvacrol, which is a powerful antiseptic. Carvacrol is a phenolic monoterpenoid found in essential oils of oregano, thyme, pepperwort, wild bergamot, and other plants. In small quantities, the carvacrol is capable of eliminating a wide variety of pathogens such as bacteria, fungi, parasites and viruses. Another advantage of its use is that those pathogens cannot create immunity and side effects, and the possibility of creating virulent mutations of bacteria and fungi is eliminated. Experiments have repeatedly confirmed the efficacy of carvacrol against Salmonella, e. Coli, Campylobacter jejuni, and Listeria. When tested on Salmonella-infected celery, carvacrol killed Salmonella colonies immediately. In one embodiment, the carvacrol may be distributed throughout the carbon filter 370 during its formation, and in another embodiment, the carbon filter undergoes surface impregnation with carvacrol particulates to achieve a fine distribution throughout its extent. The impregnation process for the activated carbon may be accomplished by placing the carbon in a tank without oxygen, heating to about 600-900 degrees Celsius, then introducing the carvacrol into the tank, which may then be superheated to a temperature ranging being between 600-1200 degrees Celsius. The impregnated active carbon used for the carbon filter 370 may be obtained from manufacturers of activate carbon, including, but not limited to: Carbon Activated Corp., which is located in Compton, Calif.

The four filters 350/370/360/390 may be secured with respect to each other to form the filter pack assembly 340 using any suitable means known in the art, including, but not limited to, a metallic band/frame, a plastic band/frame, a rubber band, adhesive, mechanical fasteners, etc. Merely to be exemplary, the four filters 350/370/360/390 are shown secured with respect to each other in FIG. 17 and FIG. 19 using a plurality of brackets 341 (e.g., two brackets per each side), which brackets may be fixedly secured to each filter in any suitable manner (e.g., mechanical fasteners, welding, etc.). The four filters 350/370/360/390 of the filter pack assembly 340 may be in contact with each adjacent filter, or may instead have a small gap therebetween, being a gap of 1 mm to 5 mm in one embodiment, a gap of 5 mm to 50 mm in another embodiment, a gap of 100 mm to 100 cm in another embodiment, and in other embodiments, a combination of such ranges or other ranges for the gap may be used. A smaller gap permits a smaller overall envelope for the housing. The entire periphery of the filter pack assembly 340 is surrounded by a gasket 341, as seen in FIG. 20 (note the gasket 341 is not illustrated on the near side of the filter pack assembly 340 shown in FIG. 17 and FIG. 19 merely to show the positioning of the different filters). The gasket may be formed of any suitable material that may inhibit airflow, including, but not limited to: rubber, cork, Teflon, foam, felt, etc. The gasket 341 may be a single piece that may be stretched over and engage the entire periphery of each of the four filters, or it may be formed in four pieces that are secured to each other and to the filters. The filter pack assembly 340 may be slidably received in a channel member within the housing 320, which channel may consist of a pair of outstanding flanges 320F that may be formed on two interior sides of the housing (FIG. 17), or on three interior sides (FIG. 18), or even on four sides (e.g., including a pair of flanges on three sides of the housing 320 and a pair of flanges on the cover plate 321). The cover plate 321, as seen in FIG. 17A, may be releasably secured to the housing 320 using mechanical fasteners (e.g., four screws), and which cover plate may be removed to provide access to the filter pack assembly 340 so it can be replaced, which should usually occur about once a year. The bulbs 381/382 of the lamp 380 are also preferably replaced at the same time. This arrangement with the plurality of filters being conjoined together into the filter pack 340 is advantageous, as it makes servicing of the air cleaning and purifying apparatus 300 easier and practical, particularly when the device is installed on the roof of an elevator car. When the cover plate 321 is secured over the access opening of the housing 320, it may seal against the gasket 341, and cause the other side of the gasket to seal against the side of the housing that is opposite the access opening (note that the gasket 341 seals against the other two sides of the interior of the housing when it is inserted in between the flanges 320F).

With the four filters 350/370/360/390 of the filter pack assembly 340 positioned as shown in FIG. 17 for the apparatus 300, a small cavity may be formed between the fan(s) 330 and the filter 350, and the air directed into the cavity through housing openings 320i may be driven through the different filters for filtering of the air according to each of the respective filter constructions. As the air exits the filter pack assembly 340 from filter 390, it may circulate in the space surrounding the lamp 380, being cleaned therein, before being forced out of the housing 320 through the exit openings 320x. The air while remaining in the housing cavity may be disinfected by the combination of the UV light from lamps 380 acting directly upon it, and by the action of the hydroxyl radicals (OH molecules) that are emitted by the titanium dioxide filter as a result of the photocatalytic effect being caused by its exposure to the UV light.

The various different embodiments of the apparatus disclosed herein may be mounted to an elevator car (or other structure), which is illustrated in FIG. 2A for the device 200. The device may be installed, for example, on the top of the elevator car, and may supply treated air into the car from the outlet openings of the apparatus. The inlet openings of the device 200 may receive air from the elevator shaft, or alternatively may receive air from a duct that is in fluid communication with the elevator car, so that the apparatus serves to continuously treat the air contained within the elevator car, which air may be cycled with respect to the air in the building environment when the elevator doors are opened for passenger ingress and egress.

While illustrative implementations of one or more embodiments of the disclosed apparatus are provided hereinabove, those skilled in the art and having the benefit of the present disclosure will appreciate that further embodiments may be implemented with various changes within the scope of the disclosed apparatus. Other modifications, substitutions, omissions and changes may be made in the design, size, materials used or proportions, operating conditions, assembly sequence, or arrangement or positioning of elements and members of the exemplary embodiments without departing from the spirit of this invention.

Accordingly, the breadth and scope of the present disclosure should not be limited by any of the above-described example embodiments, but should be defined only in accordance with the following claims and their equivalents.

What is claimed is:

1. An air cleaning and purifying system for elevators comprising:
a housing, said housing comprising: one or more walls configured to form a substantially air tight enclosure; said one or more walls comprising: one or more air inlet openings formed in a first wall portion of said air tight enclosure, and one or more air outlet openings formed in a second wall portion of said air tight enclosure;
a fan, said fan being mounted to said first wall portion, and positioned thereon to force air into said air tight enclosure through said air inlet opening and be directed towards said plurality of air outlet openings;
a lamp, said lamp mounted within said housing and configured to emit light comprising a first wavelength configured to eliminate fungi, spores of fungi, bacteria, viruses, yeasts and protozoa;
a first filter, said first filter comprising an active carbon filter configured to absorb odors; said active carbon filter impregnated with an organic essential plant oil comprising carvacrol, to eliminate bacteria, fungi, parasites, and viruses;
a second filter, said second filter comprising: a HEPA filter; said HEPA filter comprising: a fiber mesh configured to trap particles;
a third filter, said third filter comprising: a metallic compound; and
wherein said light emitted by said UV lamp comprises a second wavelength, said second wavelength configured to cause said metallic compound of said third filter to exhibit a photocatalytic effect and emit hydroxyl radicals.

2. The air cleaning and purifying system according to claim 1, wherein said first filter is shaped to extend across an entire interior periphery of said housing at a position between said fan and said plurality of air outlet openings.

3. The air cleaning and purifying system according to claim 2, wherein said second filter is shaped to extend across an entire interior periphery of said housing at a position between said first filter and said plurality of air outlet openings.

4. The air cleaning and purifying system according to claim 3,
wherein said third filter is shaped to extend across an entire interior periphery of said housing at a position between said second filter and said plurality of air outlet openings.

5. The air cleaning and purifying system according to claim 4,
wherein said lamp is positioned between said third filter and said plurality of air outlet openings.

6. The air cleaning and purifying system according to claim 1, wherein said first filter is shaped to extend across an entire interior periphery of said housing at a position between said fan and said plurality of air outlet openings.

7. The air cleaning and purifying system according to claim 6, wherein said second filter is shaped to extend across an entire interior periphery of said housing at a position between said fan and said first filter.

8. The air cleaning and purifying system according to claim 7, wherein said third filter is shaped to extend across an entire interior periphery of said housing at a position between said fan and said second filter.

9. The air cleaning and purifying system according to claim 8,
wherein said lamp is positioned between said fan and said third filter.

10. The air cleaning and purifying system according to claim 1, wherein said first wall portion and said second wall portion are substantially parallel.

11. The air cleaning and purifying system according to claim 10, wherein said first filter, said second filter, and said third filter are each substantially parallel to said first wall portion.

12. The air cleaning and purifying system according to claim 11, wherein said fan is mounted to said first wall portion to direct the air substantially perpendicularly to said first filter.

13. The air cleaning and purifying system according to claim 1, wherein said housing is made of an austenitic chrome nickel stainless steel containing molybdenum.

14. The air cleaning and purifying system according to claim 1, wherein said first and second wavelengths of light emitted by said lamp to eliminate fungi, spores of fungi, bacteria, viruses, yeasts and protozoa comprises: a wavelength in the range of 100 nanometers to 400 nanometers.

15. The air cleaning and purifying system according to claim 1, wherein said first wavelength of light emitted by said lamp to eliminate fungi, spores of fungi, bacteria, viruses, yeasts and protozoa comprises: a wavelength of about 253.7 nanometers.

16. The air cleaning and purifying system according to claim 1, wherein said first wavelength of light emitted by said lamp to eliminate fungi, spores of fungi, bacteria, viruses, yeasts and protozoa comprises: a wavelength of about 253.7 nanometers.

17. The air cleaning and purifying system according to claim 1, wherein said first wavelength of light emitted by said lamp to eliminate fungi, spores of fungi, bacteria, viruses, yeasts and protozoa comprises: a range of wavelengths between and including 222 nm and 265 nm.

18. The air cleaning and purifying system according to claim 1, wherein said metallic compound of said third filter comprises: titanium dioxide.

19. An air cleaning and purifying system for elevators comprising:
a housing, said housing comprising: one or more walls configured to form a substantially air tight enclosure; said one or more walls comprising: one or more air inlet openings formed in a first wall portion of said air tight enclosure; one or more air outlet openings formed in a second wall portion of said air tight enclosure; an access opening into said air tight enclosure; and a pair of flanges formed on opposing sides of said one or more walls of said housing;
a cover plate, said cover plate configured to releasably secure to said housing to seal said access opening and prevent air flow therethrough;
wherein said housing and said cover plate are each made of a chromium-nickel-molybdenum austenitic stainless steel;
a fan, said fan being mounted to said first wall portion, and positioned thereon to force air into said air tight enclosure through said one or more air inlet openings and be directed towards said plurality of air outlet openings;
a replaceable filter pack, said replaceable filter pack comprising:
a first filter, said first filter comprising a coarse filter configured to filter particulates being about one micrometer and larger;
a second filter, said second filter comprising an active carbon filter configured to absorb odors, said active carbon filter impregnated with an organic essential plant oil comprising carvacrol, to eliminate bacteria, fungi, parasites, and viruses;
a third filter, said second filter comprising: a HEPA filter, said HEPA filter comprising: a fiber mesh configured to trap particles being about 0.2 micrometers and larger,
a fourth filter, said fourth filter comprising: titanium dioxide;
means for securing each of said first filter, said second filter, said third filter, and said fourth filter in a fixed relation to each other, and in sequence;
a gasket, said gasket configured to surround an entire periphery of each of said first filter, said second filter, said third filter, and said fourth filter;
a lamp, said lamp mounted within said housing and configured to emit light comprising:
a first wavelength configured to eliminate fungi, spores of fungi, bacteria, viruses, yeasts and protozoa; and
a second wavelength, said second wavelength configured to cause said metallic compound of said fourth filter to exhibit a photocatalytic effect and emit hydroxyl radicals; and
wherein said first and second wavelengths of light emitted by said lamp comprise: a wavelength in the range of 100 nanometers to 400 nanometers;
wherein said replaceable filter pack is received within and supported by said pair of flanges of said housing, with said fourth filter positioned adjacent to said lamp; and
wherein said gasket is shaped to extend across and contact an entire interior periphery of said housing and said cover plate, to seal and inhibit air flow with respect to said filters within said housing, except air flow through said filters.
